(19) United States
(12) Patent Application Publication
(10) Pub. No.: US 2010/0233679 A1
(43) Pub. Date: Sep. 16, 2010

(54) DETECTION OF TRUNCATION MUTATIONS IN A LARGE BACKGROUND OF NORMAL DNA

(76) Inventors: Baochuan Guo, Solon, OH (US); Xiyuan Sun, Cleveland, OH (US)

Correspondence Address: FAY SHARPE LLP, 1228 Euclid Avenue, 5th Floor, The Halle Building, Cleveland, OH, 44115, US

(21) Appl. No.: 11/909,502

(22) PCT Filed: Mar. 24, 2005
(86) PCT No.: PCT/US05/09871

§ 371 Date: May 28, 2010

(51) Int. Cl. C12Q 1/68

(52) U.S. Cl. 435/6

(57) ABSTRACT

Various methods and strategies are disclosed for detecting human tumors, and specifically colorectal tumors. Methods are also disclosed for detecting truncation mutations in a large background of wild-type DNA. And, methods for detecting AP mutations in a large background of wild-type DNA are also disclosed.

### SEQUENCE LISTING

Reference is made to a “Sequence Listing,” appendix submitted on diskette herewith. The material contained on the diskette is hereby incorporated by reference.

### BACKGROUND

The present invention relates to the area of medical diagnostics. The invention finds particular application in conjunction with the molecular diagnosis of cancers from clinical samples where mutated DNA is present in a large background of wild-type DNA, and will be described with particular reference thereto. However, it is to be appreciated that the present invention is also amenable to other like applications.

Colorectal cancer is the second deadliest cancer in USA and 40% of affected individuals will ultimately die from their cancer. Hence, early detection is critically important to the eradication of this type of cancers. Fecal occult blood tests (FOBTs) have been used to screen colorectal cancer for many years and continue to be one of the most frequently used screening tools. However, the screening sensitivity and specificity of FOBTs are marginal. Due to the performance limitations of FOBTs, sigmoidoscopy, barium enema, and colonoscopy are used as alternatives for colorectal cancer screening. In contrast to FOBTs, sigmoidoscopy can effectively detect premalignant adenomas. However, a failure to inspect the proximal colon where most of MSI-H cancers are found is a critical shortcoming of sigmoidoscopy. Barium enema procedures can display the entire colorectum, but their sensitivity and specificity are not good. Colonoscopy is considered by most to be the standard for colorectal evaluation. However, colonoscopies are an invasive procedure which must be performed by a specially trained physician and therefore it is not clear if there exists a clinical capacity to perform screening colonoscopy on a fully national level. In addition, a colonoscopy procedure has appreciable expense and has a small risk for morbidity and mortality.

Compared with FOBTs, these instrument-based screening methods, especially colonoscopy, reduce cancer mortality more effectively, but each of them has limitations and all share the compliance disincentives of unpleasant cathartic preparation, invasive instrumentation, and small risk of harm. In fact, the low patient compliance rate has been a major problem with these instrument-based methods. It was reported in 1999 that only 14% of American over age 65 were actually screened for colorectal cancer, while the American Cancer Society recommends regular colorectal screening, starting at the age of 50, using one or more of the above methods.

One of the most promising classes of new diagnostic markers utilizes mutations in oncogenes and tumor-suppressor genes, and molecular analysis has shown its potential to be a novel noninvasive screening procedure. For example, several groups have demonstrated the power of screening colorectal cancers through the molecular analysis of the specific DNA markers exfoliated into stools. Molecular stool screening offers several potential advantages over other screening methods. First, stool screening is uniquely noninvasive, and requires no unpleasant cathartic preparation. Second, stool screening can be performed on mailed-in specimens even without a mandated physician office visit. Third, the use of specific DNA mutation markers can make stool screening more attractive by improving its sensitivity and specificity while reducing the screening costs.

Among the mutations that are used as markers for screening of colorectal cancers, the mutations in adenomatous polyposis coli (APC) gene can indicate colorectal tumors at the earliest stage of disease. Mutations in this gene initiate 85% of colorectal neoplasia, thus making them the most sensitive diagnostic markers for colorectal tumors. Moreover, mutations in APC are extremely specific. Studies indicated that no APC mutations were present in any of the control samples from patients without neoplasia. However, the detection Of mutations in APC presents extraordinarily difficult technical challenges. Unlike mutations in K-ras, which have been used for most previous studies because its mutations are clustered at two codons, mutations in APC can occur virtually anywhere within 1600 codons of the gene. Moreover, the type of mutation (base substitutions or insertion or deletions of diverse length) varies greatly among tumors. Although such APC mutations can be detected in tumors, where they are present in every neoplastic cell, they are much harder to detect in fecal DNA, where they are present in a large background of wild-type DNA. Several studies have shown that stools collected from patients with colon tumors contained as few as one mutant APC gene in about 200 wild-type APC genes. Thus, any technologies for the detection of APC mutations in fecal DNA of patients with adenomas and/or cancers must be able to (1) identify the majority of the mutations, and (2) to catch mutant DNA in the presence of a 200-fold excess of wild-type DNA in order for the screening assay to be both sensitive and specific.

It is a difficult technical challenge to detect mutations in APC, because they occur virtually within a 1600 codons sequence of APC and the type of mutations varies greatly among tumors. Fortunately, virtually all APC mutations result in stop codons caused by nonsense substitutions or small, out-of-frame-deletions or insertions. APC mutations can therefore be identified using the protein truncation test (PTT), also known as the in-vitro protein synthesis assay (IVPS). In the conventional PTT, the region of a gene containing target mutations is first PCR (polymerase chain reaction) amplified using a pair of primers that incorporate into the PCR amplicons additional sequences required for efficient cell-free translation. The amplified DNA is then added to a cell-free in-vitro transcription and translation system in the presence of radioactively labeled amino acid. The expressed proteins are finally analyzed by SDS-PAGE and autoradiography, where a band of lower molecular weight indicates mutations. Conventional PTT has been widely applied to the detection of APC truncation mutations, where DNA samples from individual FAP patients, members of a FAP family, colorectal tumors, and colorectal tumor-derived cell lines were assayed. However, because of poor sensitivity, conventional PIT is not suited for the detection of APC mutations in fecal DNA, where the abundance of mutant APC is low.

Efforts were made to develop non-radioactive detection methods for PTT assays. In one approach, fluorescent lysine TRNA is added to the in-vitro protein expression system, but this method can lead to the unspecific detection of translation products including those created from secondary translation initiation. In another approach, amino-terminal tags were incorporated to the PCR primers, followed by the selective detection of the targeted proteins by western blot analysis. For example, Muller and coworkers studied several amino-tags including HA, V5, and protein-C for this application, see Kahmann S. et al. “A Non-radioactive Protein Truncation Test for the Sensitive Detection if All Stop and Frameshift Mutations”, Human Mutat. 19, 165 (2002), herein incorporated by reference. They found that western blot analysis allows the detection of one mutant APC in 40 wild type APC genes. Recently, Gite et. al. incorporated amino-tags to both N- and C-terminals of the targeted protein and measured the relative signal levels from each tag to determine the presence of shorted polypeptide produced by the chain-truncation mutation, type. Gite S. et al. “A High-Throughput Nonisotopic Protein Truncation Test:, Nat. Biotechnology, 21, 194 (2003), herein incorporated by reference. The advantage of this method is that SDS-PAGE was not needed and thus it is amenable to high-throughput applications. However, its sensitivity is poor as it can only detect 1 mutant APC in 5 normal APC genes, thus not suitable to fecal screening either.

The extremely low abundance of mutant APC in fecal DNA creates two serious problems for the PTT assay. First, wild-type APC proteins and many non-APC proteins are much more abundant than mutant APC, yielding a background that is stronger than the signal of the mutant APC expressed. As a result, the detection signal of mutant APC is often buried in SDS-PAGE, leading to false-negatives (i.e. failure to detect mutations that are present). Second, the weak signal of the mutant APC magnifies the problem of unspecific SDS-PAGE detection of translation products including those that derive from secondary translation initiation and unspecific incorporation of the detection tag, leading to false-positives (i.e. detection of mutations when none are present).

Vogelstein et al. have used a digital protein truncation assay for the detection of colorectal tumor in fecal DNA. Traverso G. et al. “Detection of APC Mutations in Fecal DNA From Patients with Colorectal Tumors”, N. Engl. J. Med. 346, 311 (2002), herein incorporated by reference. In this digital PTT assay, every patient sample was first divided into multiple fractions so that each fraction will have a few copies of DNA, thereby increasing the mutant to normal DNA ratio. Thereafter, each of the fractions was analyzed by conventional PTT individually, from which the presence of mutant APC were identified. With this approach, they were able to detect 1 mutant APC DNA in about 500 copies of wild-type APC DNA. Recently, their original digital truncation strategy has been superseded by the addition of antigenic tags and FL-labeled amino acids. Digital protein truncation is sensitive, but it has two inherent problems. First, even with the most reliable thermostable polymerases, errors incorporated during PCR result in truncating mutations in a substantial fraction of reactions when PCR products are greater than 1 kb in size. This problem magnifies in digital PCR where only one or no mutant DNA is present in each fraction and thus errors in the first few PCR cycles can result in false positives to an unacceptable level from the stool DNA of individual without colorectal neoplasia. Second, digital protein truncation inherently requires analyses of many fractions to screen one single person and thus its total cost of screening one person could significantly increase to be unacceptable, in which the digital PCR assay could be as expensive as colonoscopy. As a result, this method may not be cost-effective when applied to clinical diagnosis.

Accordingly, there is a clear need in the art for a sensitive and specific method for detecting APC truncation mutations in a large background of wild-type DNA.

The present invention contemplates a new and improved technique for detecting truncation mutations which overcomes the above-referenced problems and others. In addition, other new techniques and strategies are provided for detecting tumors, in a noninvasive fashion, and specifically colorectal tumors, in such fashion. Moreover, methods for detecting truncation mutations in a large background of wild-type DNA are provided, and specifically APC mutations.

### BRIEF DESCRIPTION

It is an object of the invention to provide a method for detecting tumors in a human.

It is still another object of the invention to provide a method for detecting truncation mutations in a large background of wild-type DNA.

It is yet another object of the invention to provide a method for detecting colorectal tumors, and specifically, in a noninvasive manner.

It is another object of the invention to provide a method for detecting APC mutations in a large background of wild-type DNA.

These and other objects of the invention are provided by one or more of the embodiments described herein.

In one aspect according to the present invention, a method is provided for detecting truncation mutations in a large background of wild-type DNA. The method comprises providing a DNA sample including target sequences of a gene and non-target species. The method also comprises forming targeted sequence templates by amplifying some or all portions of the gene in the DNA sample and incorporating the sequences of at least two types of molecular tag. The method also comprises producing polypeptide products from the templates in which the products include mutated species of interest and wild species. The mutated species of interest include at least one type of molecular tag and the wild species include at least two types of molecular tag. The method also comprises performing a first discrimination operation in which either (i) the non-target species are distinguished from the polypeptide products, or (ii) the mutated species of interest are distinguished from the wild species. The method also comprises performing a second discrimination operation in which the other of (i) or (ii) is performed. The method comprises performing a third discrimination in which the non-target species are further distinguished from the polypeptide products. And, the method comprises analyzing the mutated species of interest.

In another aspect according to the present invention, a method is provided for detecting truncation mutations in a large background of wild-type DNA. The method comprises providing a human DNA sample including target sequences of a gene. The method also comprises forming targeted sequence templates by PCR amplifying some or all portions of the gene in the DNA sample and incorporating the sequences of a first N-terminal tag, a C-terminal tag, and a second N-terminal tag. The method also comprises producing polypeptide products from the templates in cell-free translation synthesis. The polypeptide products in the mutant form include the first and second N-terminal tags. The polypeptide products in the wild-type form include the first N-terminal tag, the C-terminal tag, and the second N-terminal tag. The method also comprises isolating the polypeptide products from other molecules of the translation system using the first N-terminal tag. The method further comprises depleting the polypeptide products in the wild-type form to enrich the polypeptide products in the mutation form using the C-terminal tag. The method still comprises further isolating the polypeptide products from other molecules of the translation system using the second N-terminal tag. The method also comprises analyzing the polypeptide products to determine the size of the polypeptide products. A truncated polypeptide product indicates a mutation in the gene in the DNA sample.

In another aspect according to the present invention, a method is provided for detecting truncation mutations in a large background of wild-type DNA. The method comprises providing a human DNA sample including target sequences of a gene. The method also comprises forming targeted sequence templates by PCR amplifying some or all portions of the gene in the DNA sample and incorporating the sequences of a N-terminal tag, a C-terminal tag, and a N-terminal reporter tag. The method also comprises producing polypeptide products from the templates in cell-free translation synthesis. The polypeptide products in the mutant form include the two N-terminal tags. The polypeptide products in the wild-type form include the N-terminal tag, the C-terminal tag, and the N-terminal reporter tag. The method also comprises isolating the polypeptide products from other molecules of the translation system using the N-terminal tag. The method further comprises depleting the polypeptide products in the wild-type form to enrich the polypeptide products in the mutation form using the C-terminal tag. The method also comprises analyzing the polypeptide products to determine the size of the polypeptide products and using the N-terminal reporter tag to further discriminate other molecules of the translation system. A truncated polypeptide product indicates a mutation in the gene in the DNA sample.

In another aspect according to the present invention, a method is provided for detecting truncation mutations in a large background of wild-type DNA. The method comprises forming targeted gene templates by PCR amplifying some or all portions of a gene in a DNA sample of a human. The method employs two runs of PCR to introduce the sequences of said N-terminal tag and N-terminal reporter tag, respectively. The templates also contain the sequences of a N-terminal tag, C-terminal tag, and a N-terminal reporter tag. The method also comprises producing polypeptide products from the templates in cell-free translation synthesis. The polypeptide products have the N-terminal tag, C-terminal tag, and N-terminal reporter tag. The method further comprises isolating the polypeptide products from other molecules of the translation system using the N-terminal tag. The method additionally comprises depleting polypeptide products in the wild-type form to enrich polypeptide products in the mutation form using the C-terminal tag. The method also comprises analyzing the polypeptide products to determine the size of the polypeptide products and using the N-terminal reporter tag to further discriminate other molecules of the translation system. A truncated polypeptide product indicates a mutation in the gene in the DNA sample.

In yet another aspect according to the present invention, a method is provided for detecting truncation mutations in a large background of wild-type DNA. The method comprises forming targeted gene templates by PCR amplifying some or all portions of a gene in a DNA sample of a human and incorporating the sequences of a N-terminal tag and a C-terminal tag. The method also comprises producing polypeptide products from the templates in cell-free translation synthesis. The polypeptide products in the mutant form comprise the N-terminal tag, The polypeptide products in the wild-type form include the N-terminal tag and the C-terminal tag. The method also comprises isolating the polypeptide products from other molecules of the translation system using the N-terminal tag. The method further comprises depleting the polypeptide products in the wild-type form to enrich polypeptide products in the mutation form using the C-terminal tag. The method still comprises the second round of isolating the polypeptide products from other molecules of the translation system using the same N-terminal tag. And, the method comprises analyzing the polypeptide products to determine the size of the polypeptide products. A truncated polypeptide product indicates a mutation in the gene in the DNA sample.

In yet another aspect according to the present invention, a method is provided for detecting truncation mutations in a large background of wild-type DNA. The method comprises forming targeted gene templates by PCR amplifying some or all portions of a gene in a DNA sample of a human. The templates also contain the sequences of a N-terminal tag and a C-terminal tag. The method also comprises producing polypeptide products from the templates in cell-free translation synthesis. The polypeptide products in the mutant form comprise the N-terminal tag, The polypeptide products in the wild-type form include the N-terminal tag and the C-terminal tag. The method also comprises isolating the polypeptide products from other molecules of the translation system using the N-terminal tag. The method additionally comprises depleting the polypeptide products in the wild-type form to enrich polypeptide products in the mutation form using the C-terminal tag. And, the method comprises analyzing the polypeptide products to determine the size of the polypeptide products and using the N-terminal tag to further discriminate other molecules of the translation system. A truncated polypeptide product indicates a mutation in the gene in the DNA sample.

In yet another aspect according to the present invention, a method for detecting truncation mutations in an APC gene, in a large background of wild-type DNA, is provided. The method comprises forming APC templates by PCR amplifying some or all portions of APC gene in a DNA sample of a human. The APC templates also contain sequences of a N-terminal tag, C-terminal tag, and a N-terminal reporter tag. The method also comprises producing polypeptide products from the APC templates in cell-free translation synthesis. The polypeptide products in the mutant form only include the N-terminal tags. The polypeptide products in the wild-type form include the N-terminal tag, C-terminal tag, and N-terminal reporter tag. The method also comprises isolating the polypeptide products from other molecules of the translation system using the N-terminal tag. The method further comprises depleting the wild-type APC polypeptide products to enrich mutated APC polypeptide products using the C-terminal tag. The method also comprises analyzing the polypeptide products to determine the size of the polypeptide products and using the N-terminal report tag to further discriminates other molecules of the translation system. A truncated polypeptide product indicates a mutation in the APC gene in the DNA sample.

The present invention provides the art with a practical, low-cost, and sensitive screening method for detecting truncation mutations in genes, such as APC, the presence of which can indicate the presence of tumors or cancers. Such method will provide clinical assays for non-invasive screening of many types of cancer including stool-based screening of colorectal cancers.

Still further advantages and benefits of the present invention will become apparent to those of ordinary skill in the art upon reading and understanding the following detailed description of the preferred embodiments.

### BRIEF DESCRIPTION OF THE DRAWINGS

The present invention may take form in various components and arrangements of components, and in various steps and arrangements of steps. The drawings are only for purposes of illustrating preferred embodiments and are not to be construed as limiting the present invention.

FIG. 1 is a flow chart diagram illustrating an embodiment of a method of the invention of detecting the truncation mutation in a large background of wild-type DNA.

FIG. 2 is a flow chart diagram illustrating an embodiment of a method of the invention of incorporating two different N-terminal and one C-terminal tags in two-run PCR.

FIG. 3 shows the western blot detection of wild-type APC with a specific peptide tag or a molecular tag added to a specific peptide.

FIG. 4 shows the western blot detection of wild-type APC with a two-tag system, where an N-terminal tag is used to remove non-APC proteins (or background bands), while another N-terminal tag was used as the reporter tag.

FIG. 5 shows the western blot detection of wild-type APC with a three-tag system, where a C-terminal tag is used to deplete wild-type APC proteins, an N-terminal tag is used to remove non-APC proteins and another N-terminal tag was used as the reporter tag.

FIG. 6 shows the detection of 1% of mutant APC DNA in 100-fold more excess of wild-type DNA using a three-tag system (an N-terminal protein C tag for removing non-APC, an N-terminal biotin report tag, and a C-terminal his tag for depleting wild-type APC.

### DETAILED DESCRIPTION OF THE PREFERRED EMBODIMENTS

The present invention provides methods for detecting tumors, and specifically colorectal tumors, by detecting truncation mutations in a large background of normal or wild-type DNA. The approach disclosed herein has several advantages over currently available methods.

The term “wild-type” refers to a gene or gene product which has the characteristics of that gene or gene product when isolated from a naturally occurring source. A wild-type gene is that which is most frequently observed in a population and is thus arbitrarily designated as the “normal” or “wild-type” form of the gene. In contrast, the term “modified” or “mutant” refers to a gene or gene product which displays modifications in sequence and/or functional properties (i.e., altered characteristics) when compared to the wild-type gene or gene product. It is noted that naturally-occurring mutants can be isolated. These are identified by the fact that they have altered characteristics when compared to the wild-type gene or gene product.

Generally, the present invention utilizes a multiple discrimination strategy to more readily detect truncation mutations in a large background of normal DNA and particularly in large backgrounds of non-target species. The multiple discrimination strategy described herein utilizes (i) three or more discrimination operations in conjunction with three or more types of molecular tags, or (ii) three or more discrimination operations in conjunction with two different types of molecular tags. A key feature of the present invention is the use of a multiple discrimination strategy as opposed to a single discrimination approach to reduce the background of non-target species.

As will be appreciated by those skilled in the art, when attempting to quantify or otherwise detect mutated proteins, polypeptides, or other species in a sample, a typical problem relates to distinguishing the mutated species from the non-mutated species, and further distinguishing the mutated species from the background of non-target molecules or species. The term “non-target” refers to molecules or species which are not of interest to the investigator.

Approaches are known for distinguishing mutated species of interest from wild species. These approaches involve the use of selective introduction of C-terminal tags to the sequences of species of interest followed by expression where the expressed wild species contains both C-terminal tags and N-terminal tags, while the expressed mutated species only contain N-terminal tags due to truncation stop caused by the mutation. Wild species are then depleted by reactions or other techniques involving the C-terminal tag to thereby increase the relative proportion of mutated species (which only contain N-terminal tags and do not contain a C-terminal tag). C-terminal tags serving this purpose are sometimes referred to herein as “C-terminal purification tags.”

Approaches are also known for distinguishing non-target molecules or species from wild species, or for distinguishing non-target molecules from wild species in conjunction with mutated species. These approaches involve the use of selective introduction of N-terminal tags to sequences of species of interest (including both the wild species and the mutated species). Expression processes can be used to produce proteins or peptides of wild and mutated species. The wild and mutated species, both containing N-terminal tags, can be distinguished from the non-target species by reactions or other techniques involving the N-terminal tags to thereby increase the relative proportion of wild species and mutated species as compared to the non-target background species. N-terminal tags serving to isolating certain species such as mutated species from non-target species before the step of analysis are sometimes referred to herein as “N-terminal purification tags”. N-terminal tags serving to identify certain species such as mutated species from non-target species during the step of analysis are sometimes referred to herein as “N-terminal reporter tags.”

As far as is known, all currently known approaches for distinguishing one or more desired species from one or more undesired species, particularly in the field of detection of very low concentrations of mutated DNA, involve single discrimination using one N-terminal tag. The present invention provides a significant advance by providing multiple discrimination strategies involving the N-terminal tag(s). This strategy enables increased detection and sensitivity particularly in applications involving large backgrounds, i.e. high populations or concentrations, of the non-target species.

In one embodiment of the invention, a method is provided for detecting truncation mutations in a large background of wild-type DNA. The method comprises various steps. First, PCR amplifies the mutation region and incorporates the sequences of purification and report tags (one N-terminal, one C-terminal purification, and one N-terminal reporter tags) to the sequence of the target gene. Second, the target gene is expressed in a cell-free in-vitro protein expression system, where the expressed wild-type protein contains both C- and N-terminal tags, while the expressed mutant protein would only have the N-terminal tags due to truncation stop created by mutations. Third, wild-type target proteins are, depleted using the C-terminal tag to enrich mutant target proteins, thus reducing the background associated with wild-type proteins. Fourth, non-target proteins are removed using the N-terminal tag to purify target proteins, thus reducing the background resulting from non-target proteins. Fifth, the purified samples are separated by SDS-PAGE, followed by western blot analysis using the N-terminal reporter tag. Western blot analysis also allows the further discrimination of the target proteins from non-target proteins. The presence of a band corresponding to shorter proteins reveals the presence of mutated DNA in the sample, thus indicating tumors.

In another embodiment of the invention, the sequences of two or more specific peptides at the same terminal are incorporated by different PCR runs to reduce the formation of non-target gene templates containing both purification and reporter tags.

In still another embodiment of the invention, the sequences of at least one N-terminal tag and regulatory region are incorporated using different PCR runs to reduce the formation of non-target gene templates containing both regulatory region and purification and reporter tags.

In yet another embodiment of this invention, all the purification and reporter tags are either the incorporated peptides themselves, or molecules added to the peptides, or the peptides chemically or biochemically modified.

In still another embodiment of the invention no less than 3 copies of mutant DNA molecules are used as PCR templates.

In another embodiment of the invention, a plurality of N-terminal tags are incorporated in a protein, peptide or species of interest, in which each of the N-terminal tags are different from one another. One or more discrimination operations such as selective reaction with one of the types of N-terminal tags, followed by selective reaction with another type of the N-terminal tags, and so on if desired, are utilized to significantly enrich the protein, peptide or species of interest as compared to the other undesired species. The undesired species are non-target species.

In another embodiment of the invention, a single type of N-terminal tag is incorporated in a protein, peptide or species of interest. A plurality of discrimination operations involving the same N-terminal tag are utilized to significantly enrich the protein, peptide or species of interest as compared to the other undesired species. The undesired species are non-target species.

In all of the various embodiments described herein, the tags, and particularly the N-terminal tags can serve as components or moieties for subsequent selective reaction in one or more discrimination operations, and can also serve as reporters for subsequent analytical or quantification operations. Specifically, for example, an N-terminal tag of interest can serve as a moiety for a subsequent selective reaction, if it is appropriately matched with one or more antibodies of interest. Once the N-terminal tag of interest is incorporated in the mutated species (and/or non-mutated species) of interest, reaction with the matching antibodies can be used to selectively enrich the reacting species in the sample.

As noted, the N-terminal tags can also serve as reporters for both analytical and discrimination operations.

Although the foregoing description of examples of tags is with regard to the N-terminal region of species of interest, the present invention also includes selective incorporation and use of certain C-terminal tags.

A variety of N-terminal tags and C-terminal tags are available which can be used for this embodiment.

Helpful background information and descriptions of particular techniques and aspects are provided in U.S. Pat. Nos. 5,709,998; 6,303,337; and “MultiColor In-Vitro Translation,” Nature Biotechnology, Vol. 21, No. 9, September 2003; all of which are hereby incorporated by reference.

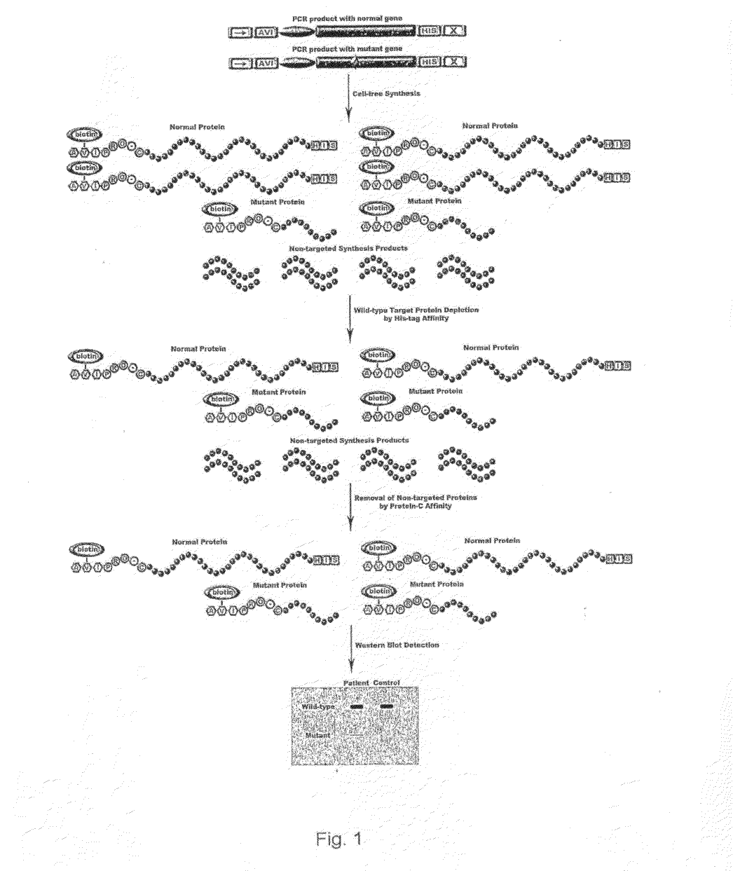

FIG. 1 shows a schematic representation of detecting a mutant gene in a large background of wild-type genes using a three-tag system. First, PCR amplifies the mutation region and incorporates the sequences of purification and report tags (one N-terminal, one C-terminal purification, and one N-terminal report tags are incorporated in the system shown in FIG. 1) to the sequence of the target gene. Second, the target gene is expressed in a cell-free in-vitro protein expression system, where the expressed wild-type protein contains both C- and N-terminal tags, while the expressed mutant protein would only have the N-terminal tags due to truncation stop created by mutations. Third, wild-type target proteins are depleted using the C-terminal tag to enrich mutant target proteins, thus reducing the background associated with wild-type proteins. Fourth, non-target proteins are removed using the N-terminal tag to purify target proteins, thus reducing the background resulting from non-target proteins. Fifth, the purified samples are separated by SDS-PAGE, followed by western blot analysis using the N-terminal reporter tag. The presence of a band corresponding to shorter proteins indicates the presence of mutated DNA in the sample, thus revealing tumors. Otherwise, no mutated DNA is called to be absent in the sample. For the purpose of illustration, the protein-C (PRO-C) peptide, biotin (which is added to the AVI peptide specifically), and His tag are used as the N-terminal purification, the N-terminal reporter, and the C-terminal purification tags, respectively.

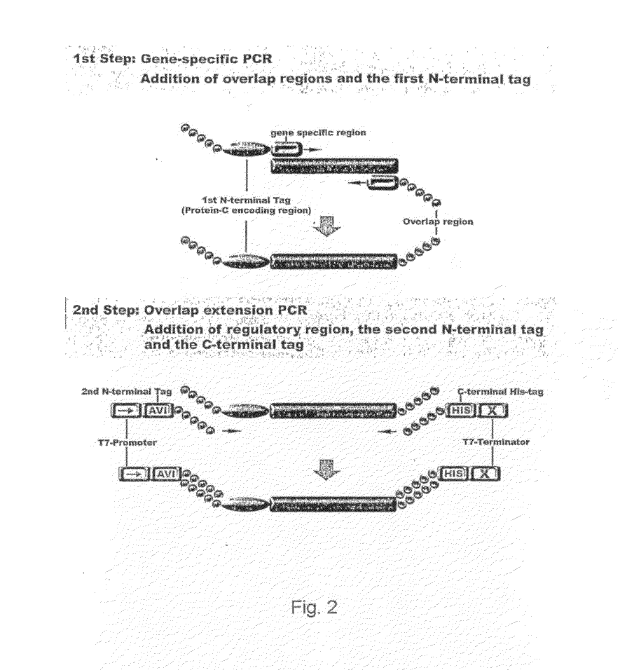

In another embodiment of the present invention, the sequences coding the specific peptides are incorporated in the sequence of the target gene by PCR. FIG. 2 displays a flow chart representing a two step PCR system to incorporate three peptide coding sequences (an N-terminal protein C tag, an N-terminal Avi tag, and a C-terminal His tag) and regulatory sequences. In another embodiment of the present invention, different coding sequences at the same terminal are incorporated in different PCR steps using different primers. In FIG. 2, a forward PCR primer inserts the N-terminal (protein C) tag to the target gene in the first PCR reaction, while another forward PCR primer inserts the second N-terminal tag (Avi) to the target gene sequence in the second PCR. A reverse PCR primer incorporates a C-terminal tag sequence. The target gene containing the specific peptide sequences is expressed in a cell free system.

In yet another embodiment of the present invention, the purification and reporter tags are the incorporated peptides themselves, or a molecule added to a specific peptide. For example, the protein-C peptide itself can be used as an N-terminal purification tag. A biotin can be added to an Avi peptide sequence after expression and this biotin can be used in either the purification or reporter tags. In another embodiment of this invention, an incorporated peptide can be chemically or biochemically modified after expression and the resulted modified peptide can also be used as either the purification or reporter tags.

In yet another embodiment of this invention, the N- and C-terminal tags are used to enrich mutant target proteins. The enrichment can be achieved by any method that allows the use of the N- and C-terminal tags to isolate the mutated target proteins, including but not limited to epitope-specific affinity interaction, chromatography, capillary electrophoresis, and size-exclusion. The N-terminal tag is mainly used to remove non-target proteins, as most of non-target proteins do not contain the N-terminal tags. This allows the removal of many highly abundant non-target proteins, thus reducing the background produced by them. The C-terminal tag is mainly used to deplete the wild-type target proteins. This is because only the target protein in the wild-type form contains the C-terminal tag, as the truncation stop created by mutations stops synthesis before it is added to mutant proteins. This allows the depletion of the wild-type protein using the C-terminal tag, thus minimizing the background problem created by the presence of highly abundant wild-type target proteins. As a result, a complex produced by an in-vitro protein expression system is simplified.

A purification tag facilities the separation of proteins of interest because of its selective interaction with other molecules which may be biological or non-biological in origin through a coupling agent. For example, the specific molecule to which the purification tag interacts, referred to as the acceptor molecule, could be a small organic molecule or chemical group such as a sulfhydryl group (—SH) or a large biomolecule such as an antibody. The binding is normally chemical in nature and may involve the formation of covalent or non-covalent bonds or interactions such as ionic or hydrogen bonding. The binding molecule or moiety might be free in solution or itself bound to a surface, a polymer matrix, or a reside on the surface of a substrate. The interaction may also be triggered by an external agent such as light, temperature, pressure or the addition of a chemical or biological molecule which acts as a catalyst.

Purification tags include native amino acids, non-native amino acids, amino acid derivatives or amino acid analogs in which a coupling agent is attached or incorporated. Attachment of the coupling agent to, for example, a non-native amino acid may occur through covalent interactions, although non-covalent interactions such as hydrophilic or hydrophobic interactions, hydrogen bonds, electrostatic interactions or a combination of these forces are also possible. Examples of useful coupling agents include molecules such as haptens, immunogenic molecules, biotin and biotin derivatives, and fragments and combinations of these molecules. Coupling agents enable the selective binding or attachment of newly formed proteins of interest to facilitate their detection or isolation. Coupling agents may contain antigenic sites for a specific antibody, or comprise molecules such as biotin which is known to have strong binding to acceptor groups such as streptavidin. For example, biotin may be covalently linked to an amino acid which is incorporated into a protein chain. The presence of the biotin will selectively bind only proteins of interest which incorporated such tags to avidin molecules coated onto a surface. Suitable surfaces include resins for chromatographic separation, plastics such as tissue culture surfaces for binding plates, microtiter dishes and beads, ceramics and glasses, particles including magnetic particles, polymers and other matrices. The treated surface is washed with, for example, phosphate buffered saline (PBS), to remove non-target proteins and other translation reagents and the proteins of interest isolated. In some case these materials may be part of biomolecular sensing devices such as optical fibers, chemfets, and plasmon detectors.

In addition to antibodies, other biological molecules exist which exhibit equally strong interaction with target molecules or chemical moieties. An example is the interaction of biotin and avidin. In this case, an affinity analog which contains the biotin moiety would be incorporated into the protein using the methods which are part of the present invention.

Many devices designed to detect proteins are based on the interaction of a target protein with specific immobilized acceptor molecule. Such devices can also be used to detect proteins once they contain affinity reporters such as biodetectors based on sensing changes in surface plasmons, light scattering and electronic properties of materials that are altered due to the interaction of the target molecule with the immobilized acceptor group.

Proteins of interest, including those which do not contain affinity-type reporters, may be isolated by more conventional isolation techniques. Some of the more useful isolation techniques which can be applied or combined to isolate and purify proteins of interest include chemical extraction, such as phenol or chloroform extract, dialysis, precipitation such as ammonium sulfate cuts, electrophoresis, and chromatographic techniques. Chemical isolation techniques generally do not provide specific isolation of individual proteins, but are useful for removal of bulk quantities of non-proteinaceous material. Electrophoretic separation involves placing the translation mixture containing proteins of interest into wells of a gel which may be a denaturing or non-denaturing polyacrylamide or agarose gel. Direct or pulsed current is applied to the gel and the various components of the system separate according to molecular size, configuration, charge or a combination of their physical properties. Once distinguished on the gel, the portion containing the isolated proteins removed and the proteins of interest purified from the gel. Methods for the purification of protein from acrylamide and agarose gels are known and commercially available.

In yet another embodiment of this invention, the mutated target gene is detected by SDS-PAGE followed by western blot analysis based on the affinity interaction with the second N-terminal tag (reporter tag). The presence of mutated DNA is indicated by the presence of a band corresponding to a protein smaller than the wild-type target protein. Because most of non-target proteins will not contain the N-terminal tags, a western blot analysis using the second N-terminal tag allows the further discrimination of non-target proteins, thus leading to improved detection of the mutated gene in a large background of normal DNA. When the removal of non-target proteins is extremely effective, the same N-terminal tag can be used both the purification and reporter tag without the use of the second N-terminal tag.

In another embodiment of this invention, the mutated target gene in the purified expression products is detected by mass spectrometry which separates molecules by size.

In another embodiment of this invention, the mutated target gene is detected by a method separating the purified expression products on the basis of size (molecular weight). The separation method includes, but not limited to, SDS-PAGE, gel filtration chromatography, gel electrophoresis, and capillary gel electrophoresis. For example, the purified expression products can be loaded onto a gel which may be composed of polyacrylamide or agarose (R. C. Allen et al., Gel Electrophoresis and Isoelectric Focusing of Proteins, Walter de Gruyter, New York 1984). Subsequent to loading the expression mixture, a voltage is applied which spatially separates the proteins on the gel in the direction of the applied electric field. The proteins separate and appear as a set of discrete or overlapping bands which can be visualized using a pre- or post-gel staining technique such as Coomasie blue staining. The migration of the protein band on the gel is a function of the molecular weight of the protein with increasing distance from the loading position being a function of decreasing molecular weight. The bands can be detected visually, photographically or spectroscopically.

In another embodiment of this invention, the mutated target gene in the purified expression products is detected by other protein separation methods (for example electrophoresis, isoelectric focusing, low pressure chromatography, high-performance, or fast-pressure liquid chromatography) that can distinguish mutant target specie from wild-type specie on the basis of the difference between the mutated target specie and wild-type specie in their chemical, or biological, or physical properties, or a combination of them. For example, purified expression products can be loaded onto a HPLC system to separate the mutated target proteins from the wild-type target proteins on the basis of the difference in their affinity toward the chromatographic separation materials.

Chromatographic techniques which are useful for the isolation and purification of proteins include gel filtration, fast-pressure or high-pressure liquid chromatography, reverse-phase chromatography, affinity chromatography and ion exchange chromatography. These techniques are very useful for isolation and purification of proteins or species containing selected reporters.

Another embodiment of the invention is directed to diagnostic kits or aids. Such kits may be useful as a rapid means to screen humans or other animals for the presence of certain diseases or disorders. Diseases which may be detected include infections, neoplasias and genetic disorders. Biological samples most easily tested include samples of blood, serum, tissue, urine or stool, prenatal samples, fetal cells, nasal cells or spinal fluid.

The present invention also contemplates kits to detect specific diseases such as familial adenomatous polyposis. In most of cases of familial adenomatous polyposis, the diseased tissues also contain chain terminated or truncated transcripts of the APC gene (S. M. Powell et al., N. Engl. J. Med. 329:1982-87, 1993).

### Example 1

This example demonstrates the use of a specific peptide (HA) sequence or a molecule (biotin) introduced to a specific peptide (Avi) sequence, as the N-terminal reporter tag for western blot analysis of wild-type APC protein synthesized from a cell-free system.

Templates were generated in a two-run PCR. The first PCR mixture contained 1×PCR buffer (Invitrogen), 0.08 u/μl of high fidelity platinum Tag DNA polymerase (Invitrogen), 0.8 pmol/μl of each APC specific primer, and up to 100 ng of wild-type human genomic DNA as templates. After an initial cycle of denaturation at 94° C. for 2 minutes, amplification was performed as follows: 30 cycles of denaturation at 94° C. for 30 seconds, annealing at 58° C. for 30 seconds, and extension at 70° C. for 1 minute. Primers were 5′-CGC TTA ATT AAA CAT ATG ACC tct gga caa agc agt aaa acc g-3′ (forward) (SEQ ID NO: 1) and 5′-TT AGT TAG TTA CCG GAT CCC TTA acg tga tga ctt tgt tgg cat ggc-3′ (reverse) (SEQ ID NO: 2), respectively. Each primer has an APC specific sequence (Bold) and an overlap region (Cap). The overlap sequences were for the second PCR reaction. In the second PCR reaction, a specific N-terminal peptide tag sequence, a promoter sequence, and a terminator sequence were introduced using either RTS Avi-Tag linear template kit or HA linear template (Roche Applied Science, Penzberg, Germany). 2.5 μl of the product of the first PCR reaction were used as templates of the second PCR reaction and the high fidelity platinum Tag was used as the DNA polymerase (Invitrogen). The second PCR (25 μl) was performed using the protocol suggested by Roche.

Protein expression (50 μl) was carried out using RTS100 E. coli HY kit (Roche Applied Science, Penzberg, Germany). A protein expression premix contained 12 μl of E. coli lysate, 10 μl of a reaction mix, and 12 μl of amino acids and 1 μl of methionine. The Avi-tag expression system included 10 μl of the products from the second PCR reaction, 2.5 μl of biotin energy mix, 2.5 μl of BirA enzyme and 35 μl of the protein expression premix. In this system, a biotin molecule was introduced to an Avi-tagged fusion protein via BirA after expression. The HA expression system included 10 μl of the products from the second PCR reaction, 5 μl of water, and 35 μl of the protein expression premix. The protein expression reaction was performed at 30° C. for 6 hours.

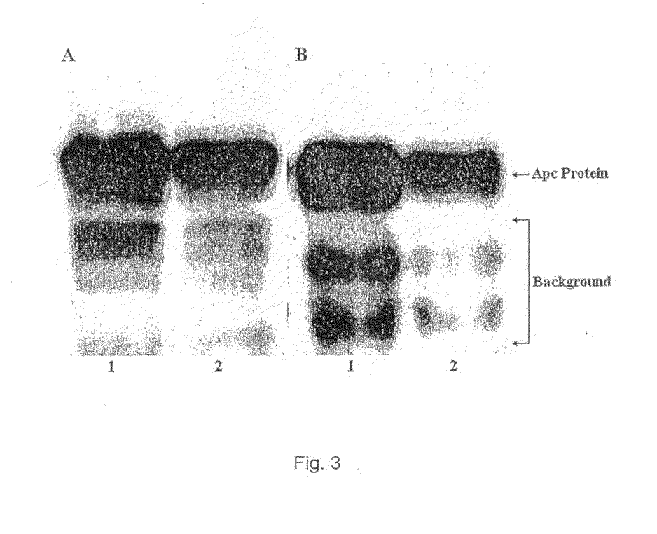

The expression products were separated using a 15% non-gradient SDS-PAGE gel. The Immobilon-P transfer membrane (Millipore, Bedford, Mass., USA) was first soaked in methanol for 1 min and then in water for 10 min before use. After electroblotting, the membrane was washed in 1×TBST for 5 minutes, then blocked 1×TBST buffer with 5% milk for 1 hour to reduce non-specific bonding. Thereafter, the membrane was incubated with Streptavidin-POD at 1:2000 (detecting biotin) or Anti-HA peroxidase at 1:4000 (detecting the HA tag) with 1×TBST buffer containing 5% milk for 1 hour at room temperature. After multiple washes, the membrane was then analyzed using chemiluminescence with the X-ray film (Kodak X-OMAT AR film) by following the manufacturers' suggested protocol. The result is displayed in FIG. 3. This example demonstrates that both a peptide sequence tag and a molecular tag introduced to a specific peptide sequence can be used as an N-terminal reporter tag. The wild-type APC protein is indicated at the right by APC. The remaining band resulted probably from those non-APC proteins (background). Specifically, in Panel A, the N-terminal tag is biotin (added to an Avi-tag). And in Panel B, the N-terminal tag is an HA tag. 10 uL of the cell-free synthesis product were loaded on Lane 1, while 5 uL of the product were loaded on Lane 2.

### Example 2

This example demonstrates the effectiveness of purification with an N-terminal tag, where an N-terminal protein-C tag was used to remove non-APC proteins (or background bands), while an N-biotin was used as the reporter tag.

Templates of APC containing an N-terminal purification tag (protein-C) and an N-terminal reporter (Avi) tag were generated in a two run PCR. The first PCR mixture contained 1×PCR buffer (Invitrogen), 0.08 u/μl of high fidelity platinum Tag DNA polymerase (Invitrogen), 0.8 pmol/μl of each APC specific primer, and up to 100 ng of wild-type human genomic DNA as templates. After an initial cycle of denaturation at 94° C. for 2 minutes, amplification was performed as follows: 30 cycles of denaturation at 94° C. for 30 seconds, annealing at 58° C. for 30 seconds, and extension at 70° C. for 1 minute. It is noted that in the first PCR reaction, the first N-terminal tag is introduced. The primers were 5′-CGC TTA ATT AAA CAT ATG ACC gaa gat cag gta gat cca cgg tta atc gat ggt aag tct gga caa agc agt aaa acc g-3′ (forward) (SEQ ID NO: 3) and 5′-TT AGT TAG TTA CCG GAT CCC TTA acg tga tga ctt tgt tgg cat ggc-3′ (reverse) (SEQ ID NO: 4), respectively. The forward primer includes an APC specific sequence (Bold), an overlap region (Cap) and a protein C sequence (Italic) to introduce the protein C. The reverse primer has an APC specific sequence (Bold) and an overlap region (Cap). The overlap sequences were for the second PCR reaction. The second PCR reaction and the cell-free expression were carried out under the same conditions described in Example 1. In the second PCR reaction, the second N-terminal tag sequence is introduced in addition to the promoter and terminator sequences.

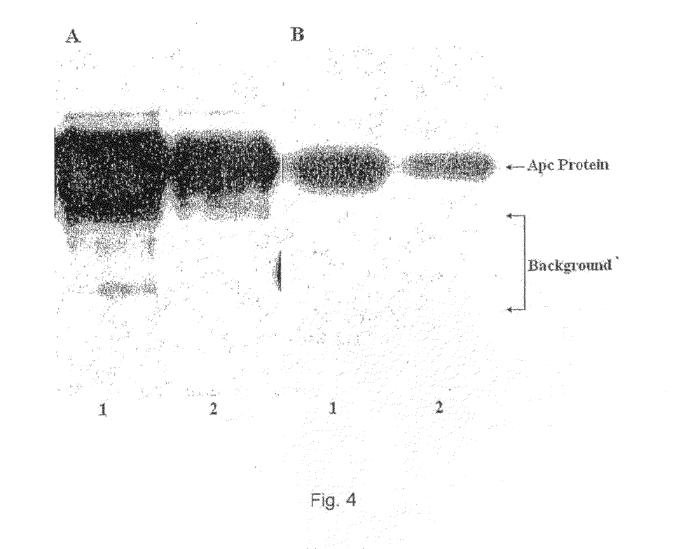

After expression, the expressed proteins were first subjected to affinity capture to remove non-APC proteins using Anti-protein C affinity matrix (Roche Diagnostics, Gmbh, Mannheim, Germany, Cat. No. 1815024) with the manufacturers' suggested protocol. 50 μl of Anti-protein C affinity matrix was used to capture APC protein from 50 μl of expression reaction mixture. The purified protein was then subjected to western blot analysis using Streptavidin-POD. The detailed western blot analysis procedure was described in Example 1. The result was displayed in FIG. 4. Panel B shows the result of affinity purification with an N-terminal protein C tag. For comparison, Panel A shows the results without affinity purification. 10 uL of the cell-free synthesis product were used on Lane 1, while 5 uL of the product were used for Lane 2. It was seen that affinity purification (Panel B) with an N-terminal tag significantly reduced the background associated with those non-APC proteins, resulting in a much clearer band for APC proteins.

### Example 3

This example demonstrates the effectiveness of a three tag system where a C-terminal tag is used to deplete wild-type APC proteins, an N-terminal tag is used to remove non-APC proteins and another N-terminal tag was used as the reporter tag.

Templates of the APC protein containing an N-terminal purification tag (protein-C tag), an N-terminal reporter (Avi-tag) tag, and a C-terminal tag (His-tag) were generated in a two run PCR. The first PCR mixture contained 1×PCR buffer (Invitrogen), 0.08 u/μl of high fidelity platinum Tag DNA polymerase (Invitrogen), 0.8 pmol/μl of each APC specific primer, and up to 100 ng of wild-type human genomic DNA as templates. After an initial cycle of denaturation at 94° C. for 2 minutes, amplification was performed as follows: 30 cycles of denaturation at 94° C. for 30 seconds, annealing at 58° C. for 30 seconds, and extension at 70° C. for 1 minute. It is noted that in the first PCR reaction, the first N-terminal tag is introduced. The primers were 5′-CGC TTA ATT AAA CAT ATG ACC gaa gat cag gta gat cca cgg tta atc gat ggt aag tct gga caa agc agt aaa acc g-3′ (forward) (SEQ ID NO: 5) and 5′-TT AGT TAG TTA CCG GAT CCC TTA acg tga tga ctt tgt tgg cat ggc-3′ (reverse) (SEQ ID NO: 6), respectively. The forward primer includes an APC specific sequence (Bold), an overlap region for adding N-terminal AviTag (Cap) and a protein C sequence (Italic) to introduce the protein C. The reverse primer has an APC specific sequence (Bold) and an overlap region for adding C-terminal His Tag (Cap). The overlap sequences were for the second PCR reaction. The second PCR reaction and the cell-free expression were carried out under the same conditions described in Example 1 except that in the second PCR reaction, the second N-terminal tag sequence and a C-terminal His tag DNA were introduced in addition to the promoter and terminator sequences using His Tag terminator primer and adding both N-terminal Avi-Tag DNA and C-terminal His Tag DNA.

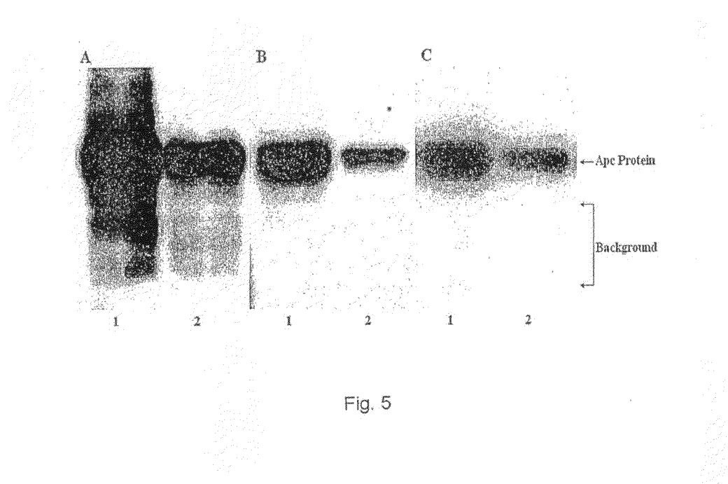

Protein expression was performed as described in Example 1. After expression, the expressed proteins were first subjected to affinity depletion to deplete wild-type APC proteins using a C-terminal His tag with Ni-NTA magnetic agarose beads (Qiagen Gmbh, Germany). Briefly, the expression product mixture were first incubated with the beads in the buffer containing 1× protein binding buffer, 50 mM NaH₂PO₄, 300 mM NaCl, and 20 mM imidazole (pH 8.0) for 3 hours at room temperature. The supernatant solution was removed and subjected to affinity purification using Anti-protein C affinity matrix to remove non-APC proteins and then subjected to western blot analysis. The detailed procedures for this affinity purification and the western blot analysis were the same as described in Example 2. The result is displayed in FIG. 5. Panel A shows the result of using a 3-tag system. For comparison, Panel B displays the results of using only one N-terminal reporter tag. Panel B shows the result after purification with the C-terminal tag, while Panel C shows the result using the C-terminal tag to first purify the expression products (mainly depleting wild-type APC proteins), followed by the purification with the N-terminal tag (removing non-APC proteins). For comparison, Panel A displays the results without any purification. 50 and 25 uL of the expression products were used for Lane 1 and 2 in all three panels, respectively. It was seen that only APC bands were observable in the western blot film and that the intensity of the wild-type APC band was significantly reduced after removing non-APC proteins and depleting wild-type APC proteins. Clearly, this example demonstrates that the western blot film became much clearer and that the intensity of the wild-type APC band was significantly reduced after removing non-APC proteins and depleting wild-type APC proteins.

### Example 4

This example demonstrates the effectiveness of using a 3 tag system to detect APC truncation mutations in a large background of 100-fold excess of wild-type DNA.

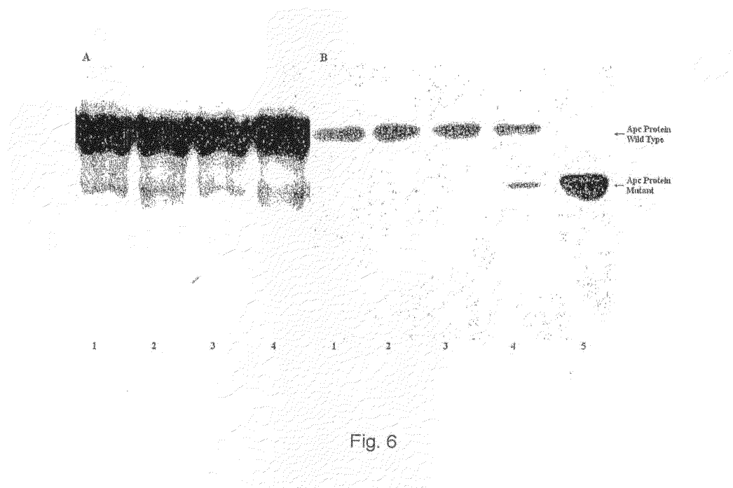

The DNA template contained 100 ng of human DNA containing 1% of mutated APC DNA collected from cell line V8-#1179b, which contained a C-to-T transition mutation in position 1 of codon 1450 of APC gene. The procedures of PCR, expression, purification, and western blot analysis were the same as described in Example 3. FIG. 6 displays the results. Panel B shows the result of affinity purification with a 3-tag system. For comparison, Panel A displays the results without affinity purification. Lanes 1 and 3 in both panels correspond to the sample containing only wild-type DNA, while Lanes 2 and 4 correspond to samples containing 1% of mutated APC DNA. Lane 5 in Panel B corresponds to the sample containing only mutated APC DNA. A total of 50 μl of the expression products was used for each lane. Clearly, this example demonstrates that with one method of this invention, it is possible to detect as little as 1% of mutated APC DNA in large background of wild-type DNA.

Although the present invention and its various aspects and embodiments have been described in terms of humans and/or human DNA, it is to be understood that the invention and accompanying claims are applicable to non-human DNA. Specifically, wide application is contemplated in the fields of veterinarian medicine and the treatment of animals such as for example, primates, dogs, equine and the like.

The present invention has been described with reference to the preferred embodiments. Obviously, modifications and alterations will occur to others upon reading and understanding the preceding detailed description. It is intended that the present invention be construed as including all such modifications and alterations insofar as they come within the scope of the appended claims or the equivalents thereof.

1. A method for detecting truncation mutations in a large background of wild-type DNA, comprising:
providing a DNA sample including target sequences of a gene and non-target sequences;
forming targeted sequence templates by amplifying some or all portions of said gene in said DNA sample, said templates including sequences of at least two types of molecular tag;
producing polypeptide products from said templates, said products including mutated species of interest and wild species, said mutated species of interest including at least one type of molecular tag, said wild species including said at least two types of molecular tag;
performing a first discrimination operation in which either (i) said non-target species are distinguished from said polypeptide products, or (ii) said mutated species of interest are distinguished from said wild species;
performing a second discrimination operation in which the other of (i) or (ii) is performed;
performing a third discrimination operation in which said non-target species are further distinguished from said polypeptide products;
analyzing said mutated species of interest on the basis of size.

2. The method of claim 1 wherein after the step of producing polypeptide products, said mutated species of interest include N-terminal tags and said wild species include N-terminal tags and C-terminal tags.

3. A method for detecting truncation mutations in a large background of wild-type DNA, comprising:
providing a human DNA sample including target sequences of a gene;
forming targeted sequence templates by PCR amplifying some or all portions of said gene in said DNA sample, said templates also including sequences of a first N-terminal tag, a C-terminal tag, and a second N-terminal tag;
producing polypeptide products from said templates in cell-free translation synthesis, said polypeptide products including said first N-terminal tag, said C-terminal tag, and said second N-terminal reporter tag;
isolating said polypeptide products from other molecules of the translation system using said first N-terminal tag;
further isolating said polypeptide products from other molecules of the translation system using said second N-terminal tag;
depleting said polypeptide products in the wild-type form to enrich said polypeptide products in the mutation form using said C-terminal tag; and
analyzing said polypeptide products to determine the size of said polypeptide products, whereby a truncated polypeptide product indicates a mutation in said gene in said DNA sample.

4. The method of claim 3 wherein the DNA sample is taken from a source selected from the group consisting of a human tumor, peripheral blood, stool, urine, bodily fluids, and combinations thereof.

5. The method of claim 3 wherein the step of forming templates employs primers which introduce a promote sequence for initiation of transcription and translation.

6. The method of claim 3 wherein the step of forming templates employs primers which introduce the sequences of a first N-terminal tag, a C-terminal tag, and a second N-terminal reporter tag.

7. The method of claim 3 wherein the cell-free translation system is selected from the group consisting of Escherichia coli lysates, wheat germ extracts, insect cell lysates, rabbit reticulocyte lysates, frog oocyte lysates, dog pancreatic lysates, human cell lysates, mixtures of purified or semi-purified translation factors and combinations thereof.

8. The method of claim 3 wherein said first N-terminal tag is at least one of a specific peptide and a molecule synthesized on the basis of said specific peptide.

9. The method of claim 3 wherein said C-terminal tag is at least one of a specific peptide and a molecule synthesized on the basis of said specific peptide.

10. The method of claim 3 wherein said second N-terminal tag is at least one of a specific peptide and a molecule synthesized on the basis of said specific peptide.

11. The method of claim 3 wherein the step of analyzing employs a method to separate said polypeptide products on the basis of size.

12. The method of claim 11 wherein said method is SDS-polyacrylamide gels, or gel filtration chromatography, or electrophoresis, or mass spectrometer, or any means to separate molecules on the basis of size.

13. A method for detecting truncation mutations in a large background of wild-type DNA, comprising:
providing a human DNA sample including target sequences of a gene;
forming targeted sequence templates by PCR amplifying some or all portions of said gene in said DNA sample, said templates also including sequences of a N-terminal tag, a C-terminal tag, and a N-terminal reporter tag;
producing polypeptide products from said templates in cell-free translation synthesis, said polypeptide products including said N-terminal tag, said C-terminal tag, and said N-terminal reporter tag;
isolating said polypeptide products from other molecules of the translation system using said N-terminal tag;
depleting said polypeptide products in the wild-type form to enrich said polypeptide products in the mutation form using said C-terminal tag; and
analyzing said polypeptide products to determine the size of said polypeptide products using said N-terminal reporter tag, whereby a truncated polypeptide product indicates a mutation in said gene in said DNA sample.

14. The method of claim 13 wherein the DNA sample is taken from a source selected from the group consisting of a human tumor, peripheral blood, stool, urine, bodily fluids, and combinations thereof.

15. The method of claim 13 wherein the step of forming templates employs primers which introduce a promote sequence for initiation of transcription and translation.

16. The method of claim 13 wherein the step of forming templates employs primers which introduce the sequences of a N-terminal tag, a C-terminal tag, and a N-terminal reporter tag.

17. The method of claim 13 wherein the cell-free translation system is selected from the group consisting of Escherichia coli lysates, wheat germ extracts, insect cell lysates, rabbit reticulocyte lysates, frog oocyte lysates, dog pancreatic lysates, human cell lysates, mixtures of purified or semi-purified translation factors and combinations thereof.

18. The method of claim 13 wherein said N-terminal tag is at least one of a specific peptide and a molecule synthesized on the basis of said specific peptide.

19. The method of claim 13 wherein said C-terminal tag is at least one of a specific peptide and a molecule synthesized on the basis of said specific peptide.

20. The method of claim 13 wherein said N-terminal reporter tag is at least one of a specific peptide and a molecule synthesized on the basis of said specific peptide.

21. The method of claim 13 wherein the step of analyzing employs SDS-polyacrylamide gels to separate said polypeptide products on the basis of size.

22. The method of claim 13 wherein the step of analyzing employs said N-terminal reporter tag to further distinguish said targeted gene from other molecules.

23. A method for detecting truncation mutations in a large background of wild-type DNA, comprising:
forming targeted gene templates by PCR amplifying some or all portions of a gene in a DNA sample of a human, said templates also containing the sequences of a N-terminal tag, C-terminal tag, and a N-terminal reporter tag;
producing polypeptide products from said templates in cell-free translation synthesis, said polypeptide products having said N-terminal tag, C-terminal tag, and N-terminal reporter tag;
isolating said polypeptide products from other molecules of said translation system using said N-terminal tag;
depleting polypeptide products in the wild-type form to enrich polypeptide products in the mutation form using said C-terminal tag; and
analyzing said polypeptide products to determine the size of said polypeptide products, whereby a truncated polypeptide product indicates a mutation in said gene in said DNA sample using said N-terminal reporter tag.

24. The method of claim 23 wherein the DNA sample is taken from a source selected from the group consisting of a human tumor, peripheral blood, stool, urine, bodily fluids, and combinations thereof.

25. The method of claim 23 wherein forming templates employs primers which introduce a promote sequence for initiation of transcription and translation.

26. The method of claim 23 wherein forming templates employs primers which introduce the sequences of a N-terminal tag, a C-terminal tag, and a N-terminal reporter tag.

27. The method of claim 23 wherein forming said templates employs two runs of PCR to introduce the sequences of said N-terminal tag and N-terminal reporter tag, respectively.

28. The method of claim 23 wherein the cell-free translation system is selected from the group consisting of Escherichia coli lysates, wheat germ extracts, insect cell lysates, rabbit reticulocyte lysates, frog oocyte lysates, dog pancreatic lysates, human cell lysates, mixtures of purified or semi-purified translation factors and combinations thereof.

29. The method of claim 23 wherein said N-terminal tag is a specific peptide or a molecule synthesized on the basis of said specific peptide.

30. The method of claim 23 wherein said C-terminal tag is a specific peptide or a molecule synthesized on the basis of said specific peptide.

31. The method of claim 23 wherein said N-terminal reporter tag is a specific peptide or a molecule synthesized on the basis of said specific peptide.

32. The method of claim 23 wherein analyzing employs SDS-polyacrylamide gels to separate said polypeptide products on the basis of size.

33. The method of claim 23 wherein analyzing employs said N-terminal reporter tag to further distinguish said targeted gene from other molecules.

34. A method for detecting truncation mutations in a large background of wild-type DNA, comprising:
forming targeted gene templates by PCR amplifying some or all portions of a gene in a DNA sample of a human, said templates also containing the sequences of a N-terminal tag and a C-terminal tag;
producing polypeptide products from said templates in cell-free translation synthesis, said polypeptide products comprising said N-terminal tag and said C-terminal tag;
isolating said polypeptide products from other molecules of said translation system using said N-terminal tag;
depleting said polypeptide products in the wild-type form to enrich polypeptide products in the mutation form using said C-terminal tag; and
analyzing said polypeptide products to determine the size of said polypeptide products, whereby a truncated polypeptide product indicating a mutation in said gene in said DNA sample using said N-terminal tag.

35. The method of claim 34 wherein the DNA sample is selected from the group consisting of a human tumor, peripheral blood, stool, urine, bodily fluids, and combinations thereof.

36. The method of claim 34 wherein forming templates employs primers which introduce a promote sequence for initiation of transcription and translation.

37. The method of claim 34 wherein forming templates employs primers which introduce the sequences of a N-terminal tag and a C-terminal tag.

38. The method of claim 34 wherein the cell-free translation system is selected from the group consisting of Escherichia coli lysates, wheat germ extracts, insect cell lysates, rabbit reticulocyte lysates, frog oocyte lysates, dog pancreatic lysates, human cell lysates, mixtures of purified or semi-purified translation factors and combinations thereof.

39. The method of claim 34 wherein said N-terminal tag is a specific peptide or a molecule synthesized on the basis of said specific peptide.

40. The method of claim 34 wherein said C-terminal tag is a specific peptide or a molecule synthesized on the basis of said specific peptide.

41. The method of claim 34 wherein analyzing employs SDS-polyacrylamide gels to separate said polypeptide products on the basis of size.

42. The method of claim 34 wherein analyzing employs said N-terminal tag to further distinguish said targeted gene from other molecules.

43. A method for detecting truncation mutations in a large background of wild-type DNA, comprising:
forming targeted gene templates by PCR amplifying some or all portions of a gene in a DNA sample of a human, said templates also containing the sequences of a N-terminal tag and a C-terminal tag;
producing polypeptide products from said templates in cell-free translation synthesis, said polypeptide products comprising said N-terminal tag and said C-terminal tag;
isolating said polypeptide products from other molecules of said translation system using said N-terminal tag;
depleting said polypeptide products in the wild-type form to enrich polypeptide products in the mutation form using said C-terminal tag;
further isolating polypeptide products from other molecules of said translation system using said N-terminal tag; and
analyzing said polypeptide products to determine the size of said polypeptide products, whereby a truncated polypeptide product indicating a mutation in said gene in said DNA sample.

44. The method of claim 43 wherein the DNA sample is selected from the group consisting of a human tumor, peripheral blood, stool, urine, bodily fluids, and combinations thereof.

45. The method of claim 43 wherein forming templates employs primers which introduce a promote sequence for initiation of transcription and translation.

46. The method of claim 43 wherein forming templates employs primers which introduce the sequences of a N-terminal tag and a C-terminal tag.

47. The method of claim 43 wherein the cell-free translation system is selected from the group consisting of Escherichia coli lysates, wheat germ extracts, insect cell lysates, rabbit reticulocyte lysates, frog oocyte lysates, dog pancreatic lysates, human cell lysates, mixtures of purified or semi-purified translation factors and combinations thereof.

48. The method of claim 43 wherein said N-terminal tag is a specific peptide or a molecule synthesized on the basis of said specific peptide.

49. The method of claim 43 wherein said C-terminal tag is a specific peptide or a molecule synthesized on the basis of said specific peptide.

50. The method of claim 43 wherein the step of analyzing employs a method to separate said polypeptide products on the basis of size.

51. The method of claim 50 wherein said method is SDS-polyacrylamide gels, or gel filtration chromatography, or electrophoresis, or mass spectrometer, or any means to separate molecules on the basis of size.

52. A method for detecting truncation mutations in an APC gene, in a large background of wild-type DNA, comprising:
forming APC templates by PCR amplifying some or all portions of APC gene in a DNA sample of a human, said APC templates also containing the sequences of a N-terminal tag, C-terminal tag, and a N-terminal reporter tag;
producing polypeptide products from said APC templates in cell-free translation synthesis, said polypeptide products including said N-terminal tag, C-terminal tag, and N-terminal reporter tag;
isolating said polypeptide products from other molecules of said translation system using said N-terminal tag;
depleting said wild-type APC polypeptide products to enrich mutated APC polypeptide products using said C-terminal tag; and
analyzing said polypeptide products to determine the size of said polypeptide products, whereby a truncated polypeptide product indicating a mutation in APC gene in said DNA sample using said N-terminal reporter tag.

53. The method of claim 52 wherein the DNA sample is from a source selected from the group consisting of a human tumor, peripheral blood, stool, urine, bodily fluids, and combinations thereof.

54. The method of claim 52 wherein forming templates employs primers which introduce a promote sequence for initiation of transcription and translation.

55. The method of claim 52 wherein forming templates employs primers which introduce the sequences of a N-terminal tag, a C-terminal tag, and a N-terminal reporter tag.

56. The method of claim 52 wherein the cell-free translation system is selected from the group consisting of Escherichia coli lysates, wheat germ extracts, insect cell lysates, rabbit reticulocyte lysates, frog oocyte lysates, dog pancreatic lysates, human cell lysates, mixtures of purified or semi-purified translation factors and combinations thereof.

57. The method of claim 52 wherein said N-terminal tag is a specific peptide or a molecule synthesized on the basis of said specific peptide.

58. The method of claim 52 wherein said C-terminal tag is a specific peptide or a molecule synthesized on the basis of said specific peptide.

59. The method of claim 52 wherein said N-terminal reporter tag is a specific peptide or a molecule synthesized on the basis of said specific peptide.

60. The method of claim 52 wherein analyzing employs SDS-polyacrylamide gels to separate said polypeptide products on the basis of size.

61. The method of claim 60 wherein analyzing employs said N-terminal reporter tag to further distinguish APC gene from other molecules.

62. The method of claim 3 wherein forming said templates employs two runs of PCR to introduce the sequences of said first N-terminal purification tag and said second N-terminal purification tag.

63. The method of claim 13 wherein forming said templates employs two runs of PCR to introduce the sequences of said N-terminal tag and said N-terminal reporter tag.

64. The method of claim 34 wherein forming said templates employs two runs of PCR to introduce the sequences of the plurality of N-terminal tags.

65. The method of claim 43 wherein forming said templates employs two runs of PCR to introduce the sequences of the plurality of N-terminal tags.

66. The method of claim 52 wherein forming APC templates employs two runs of PCR to introduce the sequences of the N-terminal tag and the N-terminal reporter tag.

67. A method for detecting truncation mutations in a large background of wild-type DNA, comprising:
forming targeted gene templates by PCR amplifying some or all portions of a gene in a DNA sample of a human, said templates also containing the sequences of an N-terminal tag, and a C-terminal tag;
producing polypeptide products from said templates in cell-free translation synthesis, said polypeptide products comprising said N-terminal tag and said C-terminal tag;
performing multiple discriminations to isolate polypeptide products from other molecules of said translation system using said N-terminal tag; and
analyzing said polypeptide products to determine the size of said polypeptide products, whereby a truncated polypeptide product indicating a mutation in said gene in said DNA sample.

68. The method of claim 67 wherein said multiple discriminations comprise three or more discrimination operations.


SEQUENCE LISTING (6 sequences)

SEQ ID NO: 1 - DNA, 43 bp, Homo sapiens
cgcttaattaaacatatgacctctggacaaagcagtaaaaccg

SEQ ID NO: 2 - DNA, 47 bp, Homo sapiens
ttagttagttaccggatcccttaacgtgatgactttgttggcatggc

SEQ ID NO: 3 - DNA, 79 bp, Homo sapiens
cgcttaattaaacatatgaccgaagatcaggtagatccacggttaatcgatggtaagtctggacaaagcagtaaaaccg

SEQ ID NO: 4 - DNA, 47 bp, Homo sapiens; identical to SEQ ID NO: 2

SEQ ID NO: 5 - DNA, 79 bp, Homo sapiens; identical to SEQ ID NO: 3

SEQ ID NO: 6 - DNA, 47 bp, Homo sapiens; identical to SEQ ID NO: 2